(19) United States
(12) Patent Application Publication
(10) Pub. No.: US 2024/0410891 A1
(43) Pub. Date: Dec. 12, 2024

(54) Method for Creating Isotope Distribution Data

(71) Applicants: Shimadzu Corporation, Kyoto-shi (JP); OSAKA UNIVERSITY, Suita-shi (JP)

(72) Inventors: Mami OKAMOTO, Kyoto-shi (JP); Yohei YAMADA, Kyoto-shi (JP); Nobuyuki OKAHASHI, Suita-shi (JP); Fumio MATSUDA, Suita-shi (JP)

(21) Appl. No.: 18/579,988

(22) PCT Filed: Jul. 19, 2021
(86) PCT No.: PCT/JP2021/027052

§ 371 Date: Jan. 17, 2024

(51) Int. Cl. G01N 33/58

(52) U.S. Cl. CPC G01N 33/58; G01N 2458/15

(57) ABSTRACT

In a method for creating isotope distribution data, samples for analysis having different concentrations of metabolites are prepared as samples containing metabolites of cells cultured in a medium containing a substrate labeled with a stable isotope, mass spectrometry is performed on each under the same analysis condition, mass spectrum data is analyzed for each to identify the type of the metabolites, and there are determined the number of metabolites included in a metabolite group made of unlabeled metabolites and/or isotopic isomers, and the signal intensities of mass peaks corresponding to all isotope isomers included in the metabolite group. The number of metabolites corresponding to all types of metabolites and the signal intensity are compared among the samples to select a sample for analysis for obtaining the isotope distribution, and data on the isotope distribution of the metabolite is integrated to create the isotope distribution data of the metabolite.

### TECHNICAL FIELD

The present invention relates to a method for creating isotope distribution data of metabolites in cultured cells.

### BACKGROUND ART

In a living organism, activities of protein change with the variation in transcription and translation of genomic DNA under the environmental influences such as diet, drug, exercise, and various types of stress. Such changes are considered to be reflected in the metabolism of various substances including low molecular weight compounds such as organic acids and amino acids in cells. Therefore, analyzing the flow (flux) of intracellular metabolism is useful for investigating the cause of a specific disease, drug discovery screening, and productivity evaluation of substance-producing cells. A series of techniques for analyzing intracellular metabolic flux is called metabolic flux analysis.

In metabolic flux analysis, in many cases, cells are cultured in a medium containing a substrate labeled with ¹³C, a stable isotope of carbon, and the culture solution is prepared to provide a sample for analysis. Then, intracellular metabolites contained in the sample are qualitatively and quantitatively measured, and based on the result, there are estimated which metabolic pathway the substrate incorporated into the cell is consumed and which substance the substrate is incorporated into. For example, in a case where cells are cultured using glucose in which the carbon at the 1st-position is substituted with ¹³C as a substrate (hereinafter, [1-¹³C] glucose), when [1-¹³C] glucose incorporated into the cell is consumed in glycolysis, pyruvate containing one ¹³C (referred to as labeled pyruvate) and pyruvate not containing ¹³C (referred to as unlabeled pyruvate) are generated in a ratio of 1:1. On the other hand, when [1-¹³C] glucose is consumed in the pentose phosphate pathway, only unlabeled pyruvate is generated. Therefore, if pyruvate is specified as one of the types of metabolites contained in the sample, and the distribution (that is, the distribution of isotopes) of the ratio of labeled pyruvate and the ratio of unlabeled pyruvate to the entire pyruvate in the sample is determined, the branching ratio between the glycolysis and the pentose phosphate pathway can be estimated.

The results of identifying the types of all metabolites contained in the sample and obtaining the isotopic distribution of various metabolites are processed using a predetermined analysis tool, which allows to provide a metabolic map schematically representing metabolic pathways. Software as an analysis tool used for metabolic flux analysis is individually developed by researchers and companies. Furthermore, in recent years, an information platform conforming to an application programming interface (API) or the like has been provided in order to make data compatible among various types of software used in the biomedical field (Non Patent Literature 1).

### CITATION LIST

### Non Patent Literature

Non Patent Literature 1: Garuda Platform, The Systems Biology Institute, specified non-profit corporation, [online], [searched on Jun. 21, 2021], Internet <http://www.garuda-alliance.org/about.html>

### SUMMARY OF INVENTION

### Technical Problem

Various types of metabolites are included in the cell. Therefore, in metabolic flux analysis, qualitative analysis and quantitative analysis of intracellular metabolites are generally performed comprehensively using a mass spectrometer. However, the intracellular content varies depending on the types of the metabolites. In addition, in order to obtain the isotope distribution for various metabolites, it is necessary to measure the amounts of a plurality of metabolites having different mass numbers depending on the incorporated stable isotopes, but the amounts of these metabolites are often greatly different. In the following description, a metabolite into which a stable isotope has been incorporated (a metabolite in which one or some of the constituent elements of the metabolite are replaced by stable isotopes) is referred to as an “isotope isomer”.

The dynamic range of the mass spectrometer limits the detectable signal intensity range, and thus it is not possible to measure the content of all metabolites contained in the sample in a single analysis. Therefore, conventionally, in a case where a peak in which the signal intensity exceeds (is saturated with) the upper limit value of quantitative measurement or a peak near the lower limit value is included in a mass spectrum obtained by performing mass spectrometry on a certain sample, the amount of metabolites has been obtained through trial and error, for example, the sample is diluted or concentrated to recreate the sample for analysis, and then mass spectrometry is performed again. Therefore, there is such a problem that it takes time to specify the types of all metabolites in the cell and determine the distribution of isotope isomers in various metabolites.

Herein, there has been described the problem in the analysis of intracellular metabolites using a mass spectrometer for the purpose of metabolic flux analysis, but there has been a similar problem in the analysis of small molecule metabolites using a mass spectrometer for the purpose of lipidomics analysis, proteomics analysis, and the like.

The objective to be solved by the present invention is to provide a method capable of rapidly obtaining data on the distribution of isotope isomers in intracellular metabolites.

### Solution to Problem

A method for creating isotope distribution data according to the present invention made to solve the above problems includes:
a preparation step of preparing a plurality of samples for analysis having different concentrations of metabolites which are obtained from cells cultured in a medium containing a substrate labeled with a stable isotope;
an analysis step of performing mass spectrometry under the same analysis condition for each of the plurality of samples for analysis;
a determination step of, for each of the plurality of samples for analysis, analyzing mass spectrum data obtained by the mass spectrometry to identify a type of the metabolite contained in each sample for analysis, and determining the number of metabolites included in a metabolite group made of unlabeled metabolites that are metabolites of the same type and in which the stable isotope is not incorporated and/or isotopic isomers that are metabolites in which one or a plurality of the stable isotopes are incorporated, and signal intensities of mass peaks corresponding to all metabolites included in the metabolite group;
a selection step of selecting a sample for analysis for obtaining an isotope distribution of each metabolite by comparing the number of metabolites included in the metabolite group corresponding to all types of metabolites and the signal intensities of mass peaks of metabolites included in the metabolite group among the plurality of samples for analysis, both the number and the signal intensities having been determined in the determination step; and
a data creation step of integrating data on an isotope distribution of the metabolite obtained by analyzing mass spectrum data of a sample for analysis selected for each metabolite to create isotope distribution data of all types of metabolites determined in the determination step.

### Advantageous Effects of Invention

The present invention can quickly provide data on the isotope distribution of intracellular metabolites as compared with a conventional method in which mass spectrometry is performed while repeating trial and error to determine the type and amount of intracellular metabolites.

### BRIEF DESCRIPTION OF DRAWINGS

FIG. 1 is a flowchart illustrating a mode of a method for creating isotope distribution data according to the present invention.

FIG. 2A is a table showing relative signal intensities of mass peaks of metabolites included in seven types of metabolite groups obtained for each sample for analysis diluted 10 fold, 100 fold, and 1000 fold.

FIG. 2B is a table showing relative signal intensities of mass peaks of metabolites included in six types of metabolite groups obtained for each sample for analysis diluted 10 fold, 100 fold, and 1000 fold.

FIG. 3 shows isotope distribution data converted for an analysis tool.

FIG. 4 is an example of a metabolic map.

FIG. 5 is a bar graph showing the ratio of the contents of metabolites included in the lactate metabolite group at 12 hours and 24 hours after the start of culture, inserted into the metabolic map.

### DESCRIPTION OF EMBODIMENTS

Hereinafter, the present invention will be described in detail.

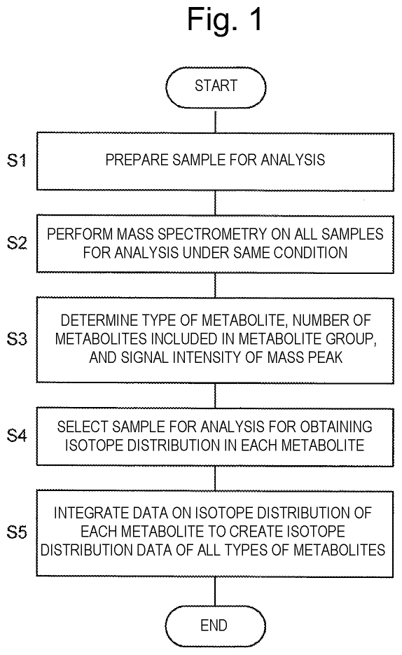

FIG. 1 is a flowchart illustrating a mode of a method for creating isotope distribution data according to the present invention. In the method for creating isotope distribution data according to the present invention, first, as a sample for mass spectrometry of a metabolite in a cell cultured in a medium containing a substrate labeled with a stable isotope, a plurality of samples for analysis having different concentrations of the metabolite are prepared (step 1). The step 1 corresponds to the preparation step of the present invention.

In the present invention, the sample to be analyzed is typically a sample derived from a living body. Examples of the sample derived from a living body include blood, tissue, and cells collected from the living body, or feces, urine, nasal discharge, saliva, and the like discharged from the inside of the living body to the outside of the living body. In addition, not only the sample derived from a living body but also water, sewage, factory effluent, and the like collected from plant bodies, soil, sea, river, lake, and the like may be used as the sample to be analyzed. Examples of the cell include microorganisms such as mold, yeast, and bacteria, or cells or tissues of multicellular organisms. In the present invention, a cell that is the sample to be analyzed itself or a cell contained in the sample to be analyzed is cultured to provide a raw material sample containing an intracellular metabolite. In this case, a culture solution is collected from a culture medium in which cells are cultured, only the cells are recovered by centrifuging the culture solution or filtering unnecessary components contained in the culture solution using a filter, intracellular metabolites are extracted using an organic solvent or the like to prepare a raw material sample, and the raw material sample is diluted or concentrated at different rates to prepare a sample for analysis. As a method for concentrating the raw material sample, it is conceivable to remove the solvent from the raw material sample by solid phase extraction or to reduce the amount of the solvent added when preparing the raw material sample. Diluting or concentrating the raw material sample can decrease or increase the concentration of the metabolite contained in the sample for analysis. When the raw material sample is prepared, as a pretreatment for mass spectrometry, a treatment for removing a substance that hinders ionization of metabolites contained in cells should be performed.

Subsequently, each of the plurality of samples for analysis is introduced into the mass spectrometer, and mass spectrometry of metabolites contained in the sample for analysis is performed under the same conditions (step 2). The step 2 corresponds to the analysis step of the present invention. The mass spectrometer is not particularly limited, but it is preferable to use a device having high resolution and capable of measuring a precise mass. Examples of such a mass spectrometer include a quadrupole mass spectrometer and a time-of-flight mass spectrometer. A liquid chromatograph mass spectrometer and a gas chromatograph mass spectrometer can also be used. As a method of ionization of the mass spectrometer, an ESI (electrospray ionization) method, an APCI (atmospheric pressure chemical ionization) method, and a MALDI (matrix-assisted laser desorption ionization) method can be used.

The stable isotope is typically ¹³C, but other examples include ²H, ¹⁵N, and ¹⁸O. The substrate labeled with a stable isotope is a compound that can be incorporated into a living body, and is a compound containing carbon, hydrogen, nitrogen, oxygen, or the like. Examples of the substrate include glucose labeled with the stable isotope ¹³C, specifically glucose in which the carbon at position 1 is substituted with ¹³C or glucose in which all carbons are substituted with ¹³C.

If cells are cultured in a medium containing a substrate labeled with a stable isotope, the substrate is taken up into the cells and consumed in metabolic pathways. As a result, a metabolite in which a stable isotope is incorporated (isotope isomer) or a metabolite in which a stable isotope is not incorporated (unlabeled metabolite) is generated, and thus a mass peak of the unlabeled metabolite and/or isotope isomer appears on a mass spectrum obtained by mass spectrometry on a sample for analysis. Typically, the mass-to-charge ratio m/z of the mass peak of the unlabeled metabolite is known. In addition, the unlabeled metabolite and the isotope isomer are different in mass number by the number of isotopes incorporated into the metabolite. Therefore, if the mass peak of the unlabeled metabolite is specified, the mass peak of the isotope isomer constituting the same metabolite as the unlabeled metabolite can be specified.

Therefore, in the method according to the present invention, mass spectrometry is performed on all the samples for analysis, and when mass spectrum data is obtained, the mass spectrum data is analyzed to specify the type of the metabolite contained in each sample for analysis, and the number of metabolites included in a metabolite group made of unlabeled metabolites and/or isotope isomers are determined, and the signal intensities of mass peaks corresponding to all the metabolites included in the metabolite group (step 3). The step 3 corresponds to the determination step of the present invention.

Subsequently, the determined number of metabolites included in the metabolite group corresponding to all types of metabolites and the determined signal intensities of mass peaks of all metabolites included in the metabolite group are compared among a plurality of the samples for analysis, and a sample for analysis for obtaining an isotope distribution of each metabolite is selected (step 4). The step 4 corresponds to the selection step of the present invention.

For example, the following criteria can be used for selecting the sample for analysis.

### <Criterion 1>

A sample for analysis in which signal intensities of mass peaks corresponding to all metabolites included in a certain metabolite group are within a predetermined range and that has the largest number of metabolites included in the metabolite group is selected as a sample for analysis for obtaining an isotope distribution of the metabolites of the metabolite group.

The predetermined range refers to detectable range set in the mass spectrometer or particularly reliable range in the detectable range.

For example, in a case where there are samples obtained by diluting the raw material sample to 10 fold, 100 fold, and 1000 fold as the sample for analysis, when there are m metabolites whose types have been specified by analyzing the mass spectrum data of each sample, it is examined whether the sample satisfying criterion 1 is the 10-fold diluted sample, the 100-fold diluted sample, or the 1000-fold diluted sample for all the m metabolite groups corresponding to the m metabolites. According to the criterion 1, a sample having a small dilution rate in the case of a metabolite having a small intracellular content tends to be selected as a sample for analysis for obtaining an isotope distribution, and a sample having a large dilution rate in the case of a sample having a large content and a relatively uniform distribution of stable isotopes tends to be selected as the sample for analysis.

### <Criterion 2>

As a result of selection based on the criterion 1, when a plurality of samples for analysis for obtaining an isotope distribution are selected, a maximum mass peak, which is a mass peak having the highest signal intensity among the mass peaks corresponding to all metabolites included in the metabolite group, is further compared among the plurality of samples for analysis, and the sample for analysis having the highest signal intensity of the maximum mass peak is selected as the sample for analysis for obtaining an isotope distribution of the metabolites of the metabolite group.

According to criterion 2, an isotope distribution in the metabolite can be obtained on the basis of the mass spectrum data having high signal intensity of the mass peak and high reliability.

### <Criterion 3>

Spectrum data selected for each metabolite group is determined such that the number of metabolite groups from which a common sample for analysis is to be selected is maximized.

In criterion 3, data on the isotope distribution of as many metabolites as possible can be created using the mass spectrum data obtained from one sample, and thus the influence of the difference in dilution rate or concentration rate on the analysis result can be suppressed.

Data on the isotope distribution of the metabolites obtained by analyzing the mass spectrum data of the sample for analysis selected for each metabolite are integrated to create isotope distribution data of all types of metabolites (step 5). The step 5 corresponds to the creation step of the present invention.

The data created by the method of the present invention can be processed, for example, in the open platform “Garuda”, and the processed result is inserted, for example, into a metabolic map schematizing metabolic pathways. A glucose metabolic system that is a basic metabolic pathway of microorganisms can be an example of the metabolic pathway included in the metabolic map. The glucose metabolic system includes a glycolytic pathway, the pentose phosphate pathway, the tricarboxylic acid cycle, and the like.

### SPECIFIC EXAMPLES

Then, the method for creating isotope distribution data according to the present invention will be described with specific examples.

Microorganisms (E. coli) were cultured in a medium containing [1-¹³C] glucose as a substrate, a culture solution was collected over time, and a cell pellet was provided by filter filtration. Then, cell constituent proteins contained in the cell pellet were extracted using an organic solvent to prepare a raw material sample, and the raw material sample was diluted 10 fold, 100 fold, or 1000 fold to provide a sample for analysis. Hereinafter, each of the samples is referred to as 10-fold diluted sample, 100-fold diluted sample, and 1000-fold diluted sample, respectively.

Subsequently, these 10-fold to 1000-fold diluted samples were analyzed by LC-MS (liquid chromatograph mass spectrometer) to provide mass spectrum data for each sample. As a result of analyzing these mass spectrum data, alanine, aspartate, glutamate, proline, glycine, serine, pyruvate, lactate, citrate, α-ketoglutarate, succinate, fumarate, and malate were identified as the types of metabolites, and mass peaks of unlabeled metabolites and isotopic isomers (metabolite groups) of these 13 metabolites were identified.

Among the 13 types of metabolites, pyruvate and lactate are representative metabolites of glycolysis, and citrate, α-ketoglutarate, fumarate, succinate, and malate are representative metabolites of the tricarboxylic acid cycle. Besides these, metabolites of glycolysis include glucose 6-phosphate, phosphoenolpyruvate, and the like. In addition, as representative metabolites of the pentose phosphate pathway described above, erythrose 4-phosphosphate, ribulose 5-phosphosphate, and the like are known. Alanine, aspartate, glutamate, proline, glycine, and serine are representative metabolites of amino acids.

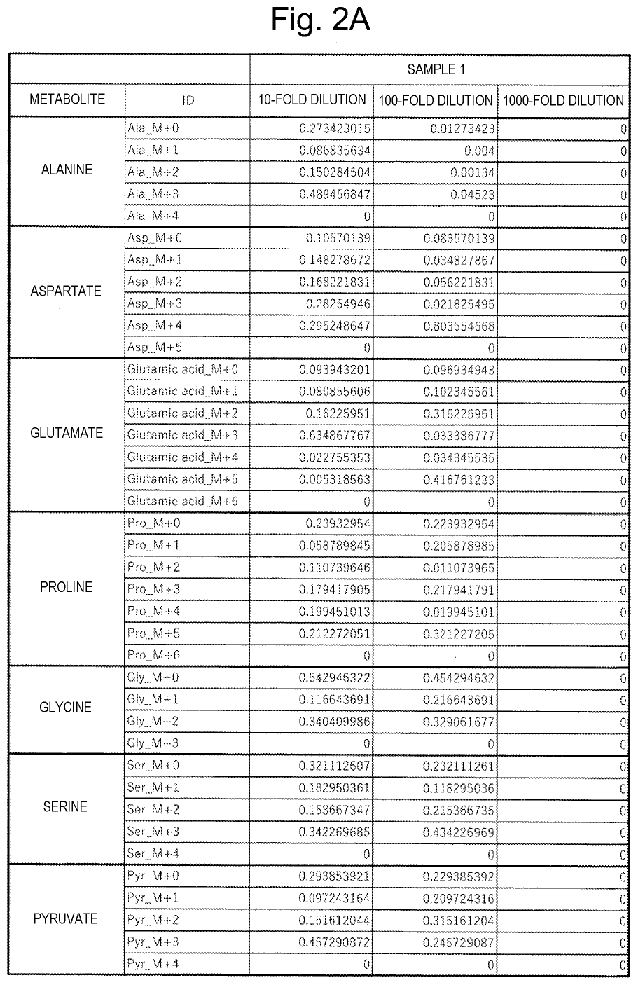

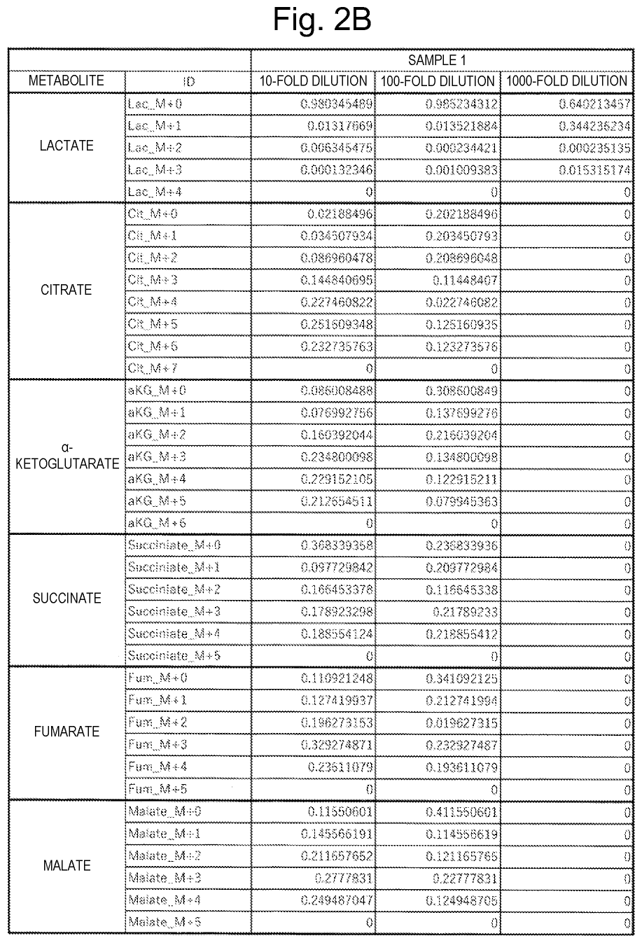

FIGS. 2A and 2B show relative values of signal intensities of mass peaks of metabolites included in the 13 metabolites obtained by analyzing mass spectrum data of each sample. In FIGS. 2A and 2B, “n” in “M+n” represents the number of ¹³C incorporated into the metabolite. That is, “M+0” represents that ¹³C is not incorporated into the metabolite, and “M+1” represents that one ¹³C is incorporated into the metabolite. Metabolites denoted by “M+0” correspond to unlabeled metabolites of the present invention, and metabolites denoted by “M+1” to “M+6” correspond to isotopic isomers.

In FIGS. 2A and 2B, the metabolite having a relative value of signal intensity of “0” is not contained in the sample or is contained in a trace amount, indicating that the signal intensity is lower than the lower limit. Therefore, it can be seen from FIGS. 2A and 2B that, for example, the metabolite group of “alanine” includes one unlabeled metabolite and three isotopic isomers, and the metabolite group of “proline” includes one unlabeled metabolite and five isotopic isomers.

In FIGS. 2A and 2B, the signal intensity of each metabolite is represented by a relative value such that the sum of the signal intensities of metabolites included in each metabolite group becomes “1”. Therefore, the isotopic distribution of each metabolite group can be found from the relative values shown in FIGS. 2A and 2B. For example, from the signal intensity of each metabolite of the 10-fold diluted sample of the metabolite group of alanine, it is found that “M+0”: 27%, “M+1”:8.6%, “M+2”: 15%, “M+3”: 49%, and “M+4”: 0%.

In the present embodiment, the analysis results are compared among the 10-fold diluted sample, the 100-fold diluted sample, and the 1000-fold diluted sample according to the criterion 1 to criterion 3 described above, and a sample for obtaining the isotope distribution is selected.

For example, in the case of the glutamate, mass peaks of six types of metabolites (one unlabeled metabolite and five isotopic isomers) were detected in the 10-fold diluted sample and the 100-fold diluted sample, whereas no mass peak was detected from the 1000-fold diluted sample. Therefore, the 1000-fold diluted sample is excluded from the selection target. In addition, no difference was observed between the 10-fold diluted sample and the 100-fold diluted sample only by comparing the numerical values shown in FIG. 2A. However, in the 100-fold diluted sample, the signal intensity of the mass peak was generally low and not stable, whereas in the 10-fold diluted sample, the signal intensity of the mass peak was reproducible. From the above, in the glutamate, the 10-fold diluted sample was adopted as the sample for determining the isotope distribution.

In addition, for example, in the case of the metabolite group of lactate, although not known only by comparing the numerical values shown in FIG. 2B, in the 10-fold diluted sample and the 100-fold diluted sample, the signal intensity of the mass peak of “M+0” exceeded the detectable range (was saturated), and thus the 1000-fold diluted sample was adopted as the sample for determining the isotope distribution.

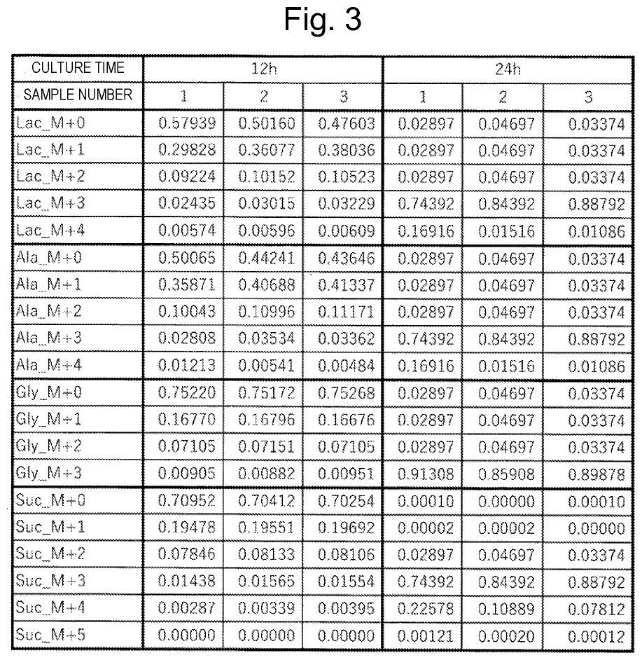

FIG. 3 shows extracted isotope distribution data of some metabolites (lactate, alanine, glycine, succinate) of the 13 types of metabolites at 12 hours and 24 hours after the start of culture. The isotope distribution data in FIG. 3 is created by selecting any one of 10-fold to 1000-fold diluted samples as samples for analysis that satisfy the above-described criterion 1, or criterion 1 and criterion 2, or criterion 3 with respect to the signal intensities of mass peaks of unlabeled metabolites and isotopic isomers included in 13 types of metabolites, and integrating data on the isotope distribution of the metabolites obtained by analyzing the respective mass spectrum data.

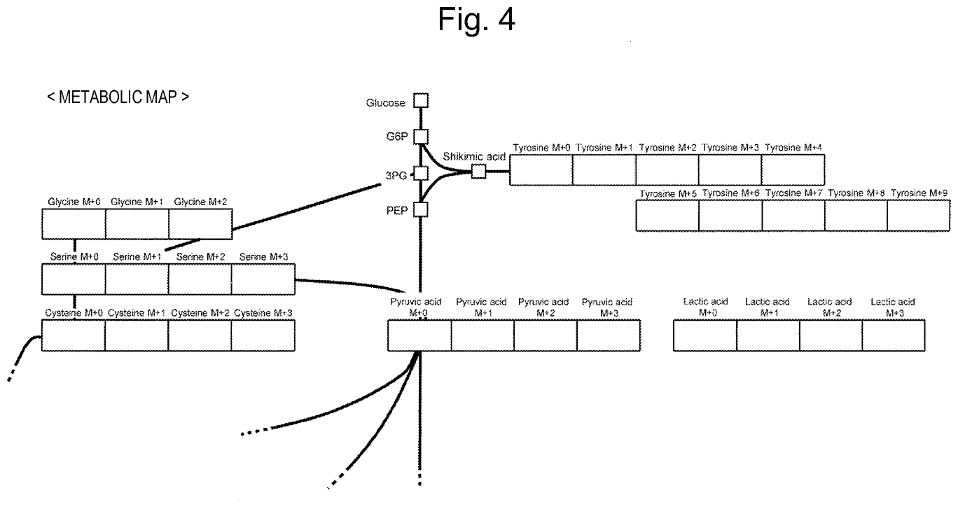

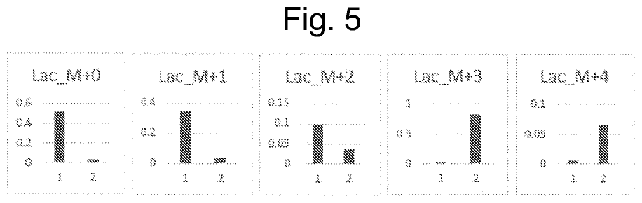

The result of analyzing the isotope distribution data shown in FIG. 3 with a predetermined analysis tool is inserted into, for example, a metabolic map as shown in FIG. 4. Examples of the analysis result inserted into the metabolic map include a bar graph as illustrated in FIG. 5. The left side of each bar graph shown in FIG. 5 shows the signal intensity (relative value) of the mass peak of lactate after 12 hours of culture, and the right side shows the signal intensity (relative value) of the mass peak of lactate after 24 hours of culture. From these bar graphs, it has been found that the content of lactate with the number of incorporated ¹³C of 0 to 2 decreased in the case of culture for 24 hours as compared with the case of culture for 12 hours, whereas the content of lactate with the number of incorporated ¹³C of 3 and 4 increased in the case of culture for 24 hours as compared with the case of culture for 12 hours. Therefore, inserting such a bar graph into the metabolic map allows visually understanding the metabolic flux.

### [Modes]

It will be understood by those skilled in the art that the exemplary embodiments described above are specific examples of the following modes.

### (Clause 1)

A method for creating isotope distribution data according to one mode of the present invention includes:
a preparation step of preparing a plurality of samples for analysis having different concentrations of metabolites as samples containing metabolites of cells cultured in a medium containing a substrate labeled with a stable isotope;
an analysis step of performing mass spectrometry under the same analysis condition for each of the plurality of samples for analysis;
a determination step of, for each of the plurality of samples for analysis, analyzing mass spectrum data obtained by the mass spectrometry to identify a type of the metabolite contained in each sample for analysis, and determining a number of metabolites included in a metabolite group made of unlabeled metabolites that are metabolites of the same type and in which the stable isotope is not incorporated and/or isotopic isomers that are metabolites in which one or a plurality of the stable isotopes are incorporated, and signal intensities of mass peaks corresponding to metabolites included in the metabolite group;
a selection step of selecting a sample for analysis for obtaining an isotope distribution of each metabolite by comparing the number of metabolites included in the metabolite group corresponding to all types of metabolites and the signal intensities of mass peaks of metabolites included in the metabolite group among the plurality of samples for analysis, both the number and the signal intensities having been determined in the determination step; and
a data creation step of integrating data on an isotope distribution of the metabolite obtained by analyzing mass spectrum data of a sample for analysis selected for each metabolite to create isotope distribution data of all types of metabolites determined in the determination step.

In the method for creating isotope distribution data of Clause 1, a plurality of samples for analysis having different concentrations of metabolites are prepared previously, and the plurality of samples for analysis are subjected to mass spectrometry under the same conditions to provide mass spectrum data. Then, the mass spectrum data obtained for each of the plurality of analysis samples is analyzed to specify the types of metabolites included in each sample for analysis, and to determine the number of unlabeled metabolites and isotopic isomers included in the metabolite group corresponding to various metabolites and the signal intensities of the mass peaks corresponding to the metabolites included in the metabolite group. An unlabeled metabolite included in a certain metabolite group can be identified from a mass peak of a known mass number observed on a mass spectrum. On the other hand, the isotope isomer can be identified from a plurality of mass peaks having different mass numbers corresponding to the number of stable isotopes unlike the mass peak of the unlabeled metabolite. When including both unlabeled metabolites and isotopic isomers, the metabolite group may include only a plurality of isotopic isomers having different numbers of stable isotopes.

When the number of metabolites included in a metabolite group corresponding to various metabolites and the signal intensities of mass peaks corresponding to the metabolites included in the metabolite group are determined, subsequently, the determined number of metabolites and the determined signal intensities are compared among the plurality of samples for analysis to select a sample for analysis for obtaining an isotope distribution of each metabolite, and to create one isotope distribution data on the isotope distribution of all types of metabolites determined in the determination step. Examples of the conditions for selecting the sample for analysis for creating the data on the isotope distribution of each metabolite include that the signal intensities of the mass peaks corresponding to all metabolites included in a metabolite group of the metabolite are within a predetermined range, that the number of metabolites included in the metabolite group is large, and that the signal intensities of the mass peaks corresponding to the metabolites included in the metabolite group have good reproducibility or are stable. In addition, a sample for analysis for obtaining the isotope distribution of each metabolite may be selected so that the number of metabolite groups from which a common sample for analysis is to be selected is maximized.

The method for creating isotope distribution data according to Clause 1 can quickly provide data on the isotope distribution of intracellular metabolites as compared with a conventional method in which mass spectrometry is performed while repeating trial and error to determine the type and amount of intracellular metabolites.

### (Clause 2)

In the method for creating isotope distribution data according to Clause 1,
the selection step may be a step of selecting a sample for analysis in which signal intensities of mass peaks corresponding to all metabolites included in a certain metabolite group are within a predetermined range and that has the largest number of metabolites included in the metabolite group as a sample for analysis for obtaining an isotope distribution of the metabolites of the metabolite group.

In the method for creating isotope distribution data according to Clause 2, using mass spectrum data of an appropriate sample for analysis according to an intracellular content, data on stable isotopes in metabolites of the sample can be obtained.

### (Clause 3)

In the method for creating isotope distribution data according to Clause 2,
the selection step may be a step of further comparing a maximum mass peak, which is a mass peak having a highest signal intensity among the mass peaks corresponding to all metabolites included in the metabolite group, among the plurality of samples for analysis, and selecting a sample for analysis having a highest signal intensity of the maximum mass peak as a sample for analysis for obtaining an isotope distribution of the metabolites of the metabolite group.

In the method for creating isotope distribution data according to Clause 3, an isotope distribution in the metabolite can be obtained on a basis of mass spectrum data having high signal intensity of mass peak and high reliability.

### (Clause 4)

In the method for creating isotope distribution data according to Clause 1,
the selection step may be a step of determining spectrum data selected for each metabolite group such that a number of metabolite groups from which a common sample for analysis is to be selected is maximized.

In the method for creating isotope distribution data according to Clause 4, data on the isotope distribution of as many metabolites as possible can be created using the mass spectrum data obtained from one sample for analysis, and thus the influence of the difference in dilution rate or concentration rate on the analysis result can be suppressed.

1. A method for creating isotope distribution data, comprising:
a preparation step of preparing a plurality of samples for analysis having different concentrations of metabolites as samples containing metabolites of cells cultured in a medium containing a substrate labeled with a stable isotope;
an analysis step of performing mass spectrometry under the same analysis condition for each of the plurality of samples for analysis;
a determination step of, for each of the plurality of samples for analysis, analyzing mass spectrum data obtained by the mass spectrometry to identify a type of the metabolite contained in each sample for analysis, and determining a number of metabolites included in a metabolite group made of unlabeled metabolites that are metabolites of the same type and in which the stable isotope is not incorporated and/or isotopic isomers that are metabolites in which one or a plurality of the stable isotopes are incorporated, and signal intensities of mass peaks corresponding to metabolites included in the metabolite group;
a selection step of selecting a sample for analysis for obtaining an isotope distribution of each metabolite by comparing the number of metabolites included in the metabolite group corresponding to all types of metabolites and the signal intensities of mass peaks of metabolites included in the metabolite group among the plurality of samples for analysis, both the number and the signal intensities having been determined in the determination step; and
a data creation step of integrating data on an isotope distribution of the metabolite obtained by analyzing mass spectrum data of a sample for analysis selected for each metabolite to create isotope distribution data of all types of metabolites determined in the determination step.

2. The method for creating isotope distribution data according to claim 1, wherein the selection step is a step of selecting a sample for analysis in which signal intensities of mass peaks corresponding to all metabolites included in a certain metabolite group are within a predetermined range and that has a largest number of metabolites included in the metabolite group as a sample for analysis for obtaining an isotope distribution of the metabolites of the metabolite group.

3. The method for creating isotope distribution data according to claim 2, wherein the selection step is a step of further comparing a maximum mass peak, which is a mass peak having a highest signal intensity among the mass peaks corresponding to all metabolites included in the metabolite group, among the plurality of samples for analysis, and selecting a sample for analysis having a highest signal intensity of the maximum mass peak as a sample for analysis for obtaining an isotope distribution of the metabolites of the metabolite group.

4. The method for creating isotope distribution data according to claim 1, wherein the selection step is a step of determining spectrum data selected for each metabolite group such that a number of metabolite groups from which a common sample for analysis is to be selected is maximized.
